(19) United States
(12) Patent Application Publication
(10) Pub. No.: US 2010/0144574 A1
(43) Pub. Date: Jun. 10, 2010

(54) Sprayable hand cleaner made of edible shellac and micellular surfactants

(76) Inventor: Michelle Diaz, Las Vegas, NV (US)

Correspondence Address: David E. Orr, 4616 NE 134th Street, Vancouver, WA, 98686, US

(21) Appl. No.: 12/316,210

(22) Filed: Dec. 10, 2008

(51) Int. Cl. A61K 8/02; A61Q 19/10

(52) U.S. Cl. 510/138; 510/140

(57) ABSTRACT

The present invention is a pressurized container from which is sprayed a gel for washing hands or other body parts. The gel, upon drying, forms a thin elastic film, to which adheres miniscule foreign matter. The gel is comprised of edible shellac and surfactants, which are maintained in the pressurized container by a solvent that evaporates, when it contacts the body part. The gel also has micellar surfactants that are activated by the person rubbing the body part where the gel is applied.

### FIELD

The present invention is a sprayable hand cleaner that when sprayed forms a thin viscoelastic film, which encapsulates a micellar surfactant solution. The thin film embeds and removes fine particles and other materials trapped within fine lines in the skin. Surfactants released when the thin film is washed off add to the cleaning efficacy of the hand cleaner.

### BACKGROUND

Hand soaps and gels are used by everyone. Of particular interest are hand cleaners used to remove grime and grit accumulated by manual labor activities.

What is needed and has not yet been provided are hand cleaners that utilize a thin film to capture fine particles that may not be subject to the action of surfactants,

The following is a list of patents and patent applications and a commercial product that relate to the field of the invention.

U.S. Pat. No. 6,426,326 discloses to liquid cleansing compositions in lamellar phase which possess a lotion-like appearance conveying signals of enhanced moisturization. However, these liquids often undergo an irreversible decrease in viscosity under freeze/thaw conditions, losing their moisturization signals. The use of low salt levels in amphoteric and anionic surfactants in a structured liquid product has been found to improve its freeze/thaw stability.

WO 01/38475 discloses a product for use in a fabric laundering process which is in the form of a self-supporting aqueous gel and which comprises one or more fabric treating agents, a gelling agent and one or more surfactants comprising a polypeptide or polysaccharide.

EP 0875236 discloses an aqueous composition for treating keratin fibers, especially human hair, which comprises natural and/or synthetic ingredients with a food or pleasurable aroma and a bitter and with a molecular weight of 250 g/mole which is 10 mg/l soluble in the formulation at 20 degrees C.

JUNGLE GEL is a commercial product which comprises: about 86.4% water; about 9.1% sodium lauryl ether sulfate; and about 1.5% PEG pentaerythrityl tetrastearate, as well as fragrances, moisturizing oils, colors, and preservatives.

Canadian Application No. 2,194,442 discloses hydrogels which are used as a suitable application form for using active substances in the treatment of skin injuries and/or for the cosmetic treatment of sensitive sites on the skin and nails. These hydrogels are sheet-like, rigid elastic structures adapted to the contours of human body sites and comprise therapeutic and/or cosmetic active substances.

U.S. Pat. No. 5,865,502 discloses aqueous, viscoelastic surfactant solutions for the cleaning of hair and skin which contain: (A) from 4 to 25% by weight of an anionic surfactant; (B) from 0 to 10% by weight of a betainic surfactant; (C) from 0 to 20% by weight of a nonionic surfactant; (D) from 0 to 6% by weight of an electrolyte; (E) from 0 to 5% by weight of a water-soluble polymer; and (F) from 0 to 5% by weight of a further constituent; in which the sum of the amounts of (A), (B), and (C) is at least 10% by weight and the sum of the amounts of (C), (D), and (E) is between 2 and 20% by weight, in each case based on the total weight of the aqueous solution, and having a shear modulus, G₀, between 50 and 500 Pa at temperatures between 20 and 40. degree. C. and a pH of from 4 to 8, and in which the conditions for the identity of the storage modulus, G′, and the loss modulus, G″, are in the angular frequency range between 0.1 and 60 rad⁻¹, exhibit optimum flow behavior for the intended applications.

In U.S. Pat. No. 4,661,359 Seaborne et. al, in Compositions and methods for preparing an edible film of lower water vapor permeability disclose edible film coating compositions of low moisture permeability and their methods of preparation. The compositions comprise cross-linked, refined shellac and hydroxypropyl cellulose (HPC) in a weight ratio of 1:0.001 to 1:2. Highly preferred compositions additionally comprise mono- or di-carboxylic acid adducts such as adipic, succinic, lauric, or stearic acids in a weight ratio of acid to combined weight of shellac and cellulose of 1:33 to 1:10. In the preferred method of preparing, the shellac is cross-linked in a dry, molten mixture with the HPC by heating at 130.degree. to 175.degree. C. for 2 to 15 minutes. The coating compound while molten is dissolved in a food grade solvent, applied to a substrate, and dried. The coating compositions are particularly useful as a moisture barrier in composite food articles having phases in contact which differ substantially in water activity. Effective films range from 0.1 to 5 mils in thickness.

### OBJECTS

The present invention has been conceived according to several benefits and objectives. A first object of the present invention is a hand cleaner that incorporates a thin film adhering mechanism to capture fine materials that are trapped in the pores and crevasses of a hand.

A second object is a cleaner that also utilizes surfactants to dissolve oils and other hydrocarbon materials.

A third object is a cleaner that may be dispensed from a pressurized vessel, or may be pumped or poured from a vessel

And yet another object is a cleaner that may be used with or without water.

Other benefits and advantages of the invention will appear from the disclosure to follow. In the disclosure reference is made to the accompanying drawings, which form a part hereof and in which is shown by way of illustration a specific embodiment in which the invention may be practiced. This embodiment will be described in sufficient detail to enable those skilled in the art to practice the invention, and it is to be understood that other embodiments may be utilized and that structural changes may be made in details of the embodiments without departing from the scope of the invention.

### SUMMARY

In an exemplary embodiment, the present invention is disclosed as a pressurized container from which is sprayed a gel for washing hands or other body parts. The gel, upon drying, forms a thin elastic film, to which adheres miniscule foreign matter. The gel is comprised of edible shellac and surfactants, which are maintained in the pressurized container by a solvent that evaporates, when it contacts the body part. The gel also has micellar surfactants that are activated by washing with water. The surfactants mix and adhere to matter by a person by rubbing the dried gel, which performs a thorough mixing action by pressure and friction.

### BRIEF DESCRIPTION OF THE DRAWINGS

FIG. 1A illustrates the general principles of operation of an aerosol dispensing vessel.

FIG. 1B and FIG. 1C depict the operation of an actuating mechanism for releasing aerosol from a pressurized container.

### DETAILED DESCRIPTION

In the figures and following description an exemplary embodiment is disclosed, the exemplary embodiment disclosed as a pressure vessel containing a gel, which dries as an elastic film upon contact with a surface. The gel is comprised of a solvent, an edible shellac or similar material, and micellar surfactants.

The invention is used by spraying the gel from a pressurized container. The gel is maintained as a solution in an appropriate solvent, that, when dries, forms a flexible film on the surface. The film is washed from the surface, and in the process of washing materials are removed from the surface.

### An Exemplary Embodiment

### Pressurized Vessel

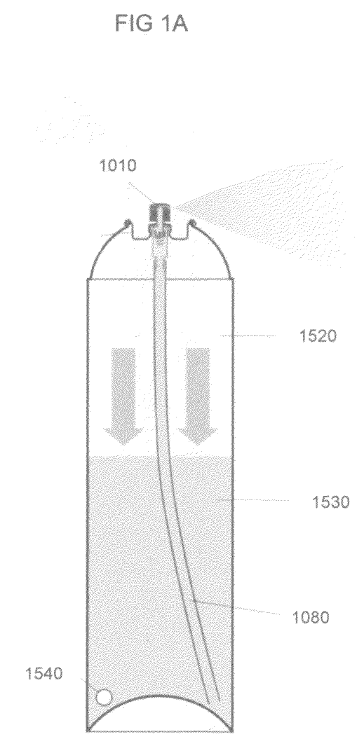

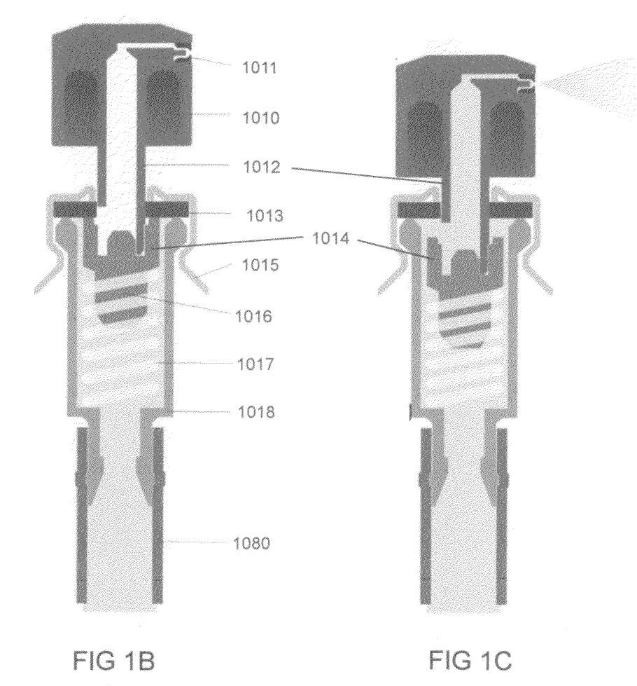

FIG. 1A shows the general structure and operation of an aerosol vessel. The following description does not restrict the invention to the structure and operation shown and described. Specifically, the aerosol may be pumped from a container or may also be poured as a gel in a more viscous form.

Typically, a vessel consists of a container having a actuator 1010 on top. The container holds a gas 1520 under pressure and a material 1530 that is to be dispensed. At the bottom of the container one generally finds a “pea” that, when agitated, prevents coagulation of the material. When the actuator 1010 is depressed the gas 1520 forces the material 1530 up into the dip tube 1080 and out the vessel as a spray.

FIG. 1B shows the actuator in detail and FIG. 1C shows the actuator when depressed. In FIG. 1B, the following components are illustrated:

1. 1011 an orifice insert though which the aerosol is sprayed

2. 1010 the actuator which is depressed

3. 1012 the stem through which the aerosol is sprayed out

4. 1013 a gasket

5. 1014 actuator valve

6. 1015 the valve cup for sealing the actuator in the vessel

7. 1016 spring cup for engaging the spring

8. 1017 spring

9. 1018 actuator housing

10. 1080 dip tube

When the actuator 1010 is depressed, the actuator valve is pressed downward permitting aerosol to pass outward as shown in FIG. 1C.

### The Gel

The gel comprises an edible film-forming material and at least one surfactant. The gel is maintained in a liquid form by a suitable solvent that dries quickly when the gel is sprayed onto a surface.

### Edible Shellac (Glazing Agent)

Shellac is soluble in alkaline solutions such as ammonia, sodium borate, sodium carbonate, and sodium hydroxide, and also in various organic solvents. When dissolved in alcohol, typically blends containing ethanol and methanol, shellac yields a coating of superior durability and hardness and is available in numerous grades. Shellac refined for industrial purposes either retains its natural wax content or is refined wax-free by filtration.

Shellac is edible and is used as a glazing agent on pills and candies. Because of its alkaline properties, shellac-coated pills may be used for a timed enteric or colonic release. It is also used to replace the natural wax of the apple, which is removed during the cleaning process.^{[8]} When used for this purpose, it has the food additive E number E904. This coating may not be considered as vegetarian as it may, and probably does, contain crushed insects. In the tablet manufacture trade, it is sometimes referred to as “beetlejuice” for this reason. In some cases, shellac is known to cause allergies on contact, resulting in skin irritations.^{[9}

Any edible food film composition providing the above attributes can be used herein, e.g., conventional shellac. Especially suitable compositions, composition preparation and application techniques for use as the film layers are disclosed in Seaborne et al U.S. Pat. No. 4,661,359, issued Apr. 28, 1987, entitled EDIBLE COATING COMPOSITIONS COMPRISING ACID CATALYZED SHELLAC AND HYROXYPROPYL CELLULOSE AND METHOD OF PREPARATION, and U.S. Pat. No. 788,178, filed Oct. 16, 1985, now Seaborne et al U.S. Pat. No. 4,710,228, entitled EDIBLE COATING COMPOSITION AND METHOD OF PREPARATION, each of which are incorporated herein by reference. The coatings there described in detail are polymers of a specially defined, shellac or copolymers of the shellac with certain other reactants having a reactive acid or hydroxyl moiety.

The specification of edible shellac should not be construed as a limitation of the present invention. Other glazing materials may be use, such as glazing agents, or polishing agents, are food additives providing shiny appearance or protective coating to foods. Mostly they are based on waxes. Examples of other possible glazing agents are: stearic acid, beeswax candelilla wax, microcrystalline wax, lanolin, oxidized polyethelyne wax and esters of colophonium.

### Surfactants

Surfactants reduce the surface tension of water by adsorbing at the liquid-gas interface. They also reduce the interfacial tension between oil and water by adsorbing at the liquid-liquid interface. Surfactants may also assemble in the bulk solution into aggregates. Examples of such aggregates are vesicles and micelles. The concentration at which surfactants begin to form micelles is known as the critical micelle concentration or CMC. When micelles form in water, their tails form a core that can encapsulate an oil droplet, and their (ionic/polar) heads form an outer shell that maintains favorable contact with water. When surfactants assemble in oil, the aggregate is referred to as a reverse micelle. In a reverse micelle, the heads are in the core and the tails maintain favorable contact with oil. Surfactants are also often classified into four primary groups; anionic, cationic, non-ionic, and zwitterionic (dual charge).

The gel comprises anionic and amphoteric surfactants, wherein the anioic surfactant comprises 2% to 20% of the gel and the amphoteric surfactant comprises from 1% to 20% of the gel by volume.

The anionic surfactant (based on sulfate, sulfonate or carboxylate anions) may be selected from: sodium dodecyl sulfate (SDS), ammonium lauryl sulfate, and other alkyl sulfate salts, sodium laureth sulfate, also known as sodium lauryl ether sulfate (SLES), and alkyl benzene sulfonate.

Zwitterionic (amphoteric) surfactants may be selected from dodecyl betaine, dodecyl dimethylamine oxide, docamidopropyl betaine, and coco ampho glycinate.

### Application and Use of the Gel

The gel may be prepared in a variety of forms having differing levels of liquidity; the liquidity level controlled by the percentage of solvents used to dissolve and suspend the surfactants and glazing agents. For example, by utilizing solvents of a particular type and quantity, the gel may be made into an aerosol for spraying. A different formulation of solvent type and amount will result in a gel in liquid suspension, which may be poured.

The gel, upon drying, adheres to the skin of the recipient of the gel, and as a result encapsulates particles on the persons skin. The person, by rubbing the area of application, accomplishes two objectives: (1) the surfactants in the gel are spread and forced into crevasses in the person's skin, thereby absorbing more detritus from the person's skin, and (2) the dried glazing agent is broken and scourged from the skin, thereby pulling the embedded detritus away from the person's skin.

What is claimed is:

1. A pressurized container from which is sprayed a gel for washing a person's hands or other body parts, the gel, upon drying, forms a thin elastic film, to which adheres miniscule foreign matter, wherein the gel comprising edible shellac and surfactants, which are maintained in the pressurized container by a solvent that evaporates, when it contacts the body part, the gel having micellar surfactants activated by washing with water, whereby the surfactants mix and adhere to matter by the person rubbing the dried gel, thereby performing a thorough mixing action by pressure and friction.
